(19) United States
(12) Patent Application Publication
(10) Pub. No.: US 2005/0107411 A1
(43) Pub. Date: May 19, 2005

(54) Method for treating circadian rhythm disruptions

(76) Inventors: Douglas J MacNeil, Westfield, NJ (US); Lauren P. Shearman, Westfield, NJ (US); Leonardus H. T. Van Der Ploeg, Scotch Plains, NJ (US)

Correspondence Address: MERCK AND CO., INC, P O BOX 2000, RAHWAY, NJ, 07065-0907, US

(21) Appl. No.: 10/497,558

(22) PCT Filed: Dec. 13, 2002
(86) PCT No.: PCT/US02/40015

Related U.S. Application Data
(60) Provisional application No. 60/342,177, filed on Dec. 17, 2001.

(51) Int. Cl. A61K 31/519; A61K 31/4747

(52) U.S. Cl. 514/267; 514/278

(57) ABSTRACT

A neuropeptide Y Y5 antagonist is useful, alone or in conjunction with other agents, for altering circadian rhythmicity and alleviating circadian rhythm disorders and for enhancing and improving the quality of sleep. The present invention further provides for the use of a medicament for carrying out these methods.

### BACKGROUND OF THE INVENTION

Circadian rhythms are exhibited by all eukaryotic plants and animals, including man. Biological rhythms are periodic fluctuations in biological processes over time, including circadian as well as seasonal variations. Circadian, or approximately 24-hour, rhythms include the production of biological molecules such as hormones, the regulation of body temperature, and behavior such as wakefulness, alertness, sleep and periods of activity. Circadian rhythms are endogenous, self-sustained oscillations over 24-hour periods found in organisms ranging from prokaryotes to humans (J S Takahashi, et al. Science, 217, 1104-1111 (1982)).

In nature, circadian rhythms are closely tied to environmental cues that impose a 24-hour pattern on many of these fluctuations. The regulation of circadian rhythms by signals from the environment involves “entrainment” of the circadian rhythm. The environmental signals which affect entrainment of the circadian rhythm are termed “zeitgebers”, an example of which is the light-dark cycle.

The control of many circadian rhythms in mammals is mediated by the portion of the brain called the suprachiasmatic nuclei (SCN). In humans as well as other mammals, the circadian clock, which controls all endogenous circadian rhythms, is located in the SCN of the hypothalamus. Activity, alertness, core body temperature, and many hormones all have endogenous circadian rhythms controlled by the SCN. The SCN is the primary pacemaker for circadian rhythms in mammals. Circadian rhythms are primarily entrained by the environmental light-dark cycle. One of the most important and reproducible characteristics of a circadian clock is that it can respond to exogenous light/dark signals. The circadian clock is composed of three parts: light-input pathways, a clock, and effector (“output”) pathways. Light signals are conveyed by the retina to the SCN, and the pineal gland produces melatonin (N-acetyl-5-methoxytryptamine), which is regulated by the SCN. Information regarding light is conveyed from the retina to the SCN via the direct retinohypothalamic tract (RHT), as well as indirectly via the lateral geniculate nucleus (LGN) (D C Klein et al., (1991) Suprachiasmatic nucleus: the mind's clock. New York: Oxford University Press).

It has been suggested in the art that excitatory amino acids are involved in the transduction of information regarding the light-dark cycle to the SCN. Acetylcholine, neuropeptide Y, GABA, 5HT₁ receptor functioning, glutamate, and substance P receptor may play a role in the entrainment and/or generation of circadian rhythms in mammals. The oscillator in the SCN can be reset by photic input, which is mediated by glutamatergic afferents originating in the retina. Glutamate can mimic the effects of light on the mammalian circadian clock in vitro. Both NMDA glutamate receptors and non-NMDA receptors can mediate the effects of light on the circadian clock (J M Ding, et al. Science, 266, 1713-1717 (1994); S. Shibata, et al. Am. J. Physiol., 267, R360-R364 (1994)). Application of NMDA in vitro can phase-shift electrical activity rhythms in hypothalamic brain slices containing the SCN. Activation of NMDA receptors is believed to be an important step in the transmission of photic information to the SCN (E M Mintz, et al., J. Neurosci., 19, 5124-30 (1999).

Neuropeptide Y (NPY) is a 36 amino acid peptide that is a member of the pancreatic polypeptide family, which also includes pancreatic polypeptide (PP) and peptide YY (PYY). NPY is located throughout the central and peripheral nervous systems and affects a diverse range of biological functions, including central endocrine secretion, vascular and smooth muscle activity, appetite, memory, anxiety, blood pressure regulation and reproduction. See, e.g., Karla, et al., Phys. & Behavior 50, 5 (1991).

NPY receptors are members of the G protein-coupled receptor superfamily. At present, NPY is known to bind to at least five receptors: Y1, Y2, Y3, Y4 and. Y5. Both Y1 and Y5 NPY receptors are expressed in the SCN (P J Larsen and P Kristensen, Mol. Brain Res. 60, 69-76 (1998). It is thought that a Y5 antagonist can prevent the effect of NPY on NMDA-induced phase shifts of the SCN circadian neural activity rhythm. The Y5 receptor mediates the blocking effect of NPY on NMDA-induced phase shifts.

The SCN and the circadian clock control the phases and rhythms of a number of hormonal rhythms in humans. One of the most well-characterized SCN outputs is to the pineal body, via a circuitous route from the hypothalamus to the spinal cord and then back to the pineal. The human pineal gland secretes melatonin in a circadian fashion, such that the plasma concentrations observed during the night are ten to forty times higher than those observed during the day. This plasma melatonin rhythm is a true circadian rhythm, and therefore not dependent upon the exogenous light-dark cycle, as it persists in blinded animals and blind humans. However, light is able to influence the endogenous melatonin rhythm. Light exposure during the night, when plasma melatonin concentrations are high, is able to rapidly suppress plasma melatonin to near daytime levels in a dose-dependent manner (C A Czeisler, et al. N. Eng. J. Med., 332, 6-11 (1995); McIntyre I M, et al. J Pineal Res, 6, 149-56 (1989); D B Boivin, et al. Nature, 379, 540-2 (1996)). The suppressive effects of light on plasma melatonin concentrations are believed to be mediated through the retina-SCN-pineal neural pathway (R Y Moore, et al. Science, 210, 1267-9 (1980)). It is believed that a NPY Y5 agonist can block the NMDA-induced phase shift (delay) of SCN circadian neuronal activity. A NPY Y5 receptor antagonist might facilitate “resetting” of the clock by inducing sleep at clock-relevant times, much like melatonin. A NPY Y5 receptor antagonist is expected to alter photic entrainment of the circadian clock in vivo.

Circadian rhythms are also an important modulator of sleep. Although sleep is necessary for survival, its precise homeostatic contribution is unknown. Sleep is not a uniform state, but rather involves several stages characterized by changes in the individual's EEG. A non rapid eye movement (NREM) type (75 to 80% of total sleep time) ranges in depth through stages 1 to 4 (deepest level). Stage 1 sleep is drowsiness, in which the EEG displays a lower voltage, more mixed frequencies and deterioration of alpha rhythm relative to the EEG when the individual is awake. In stage 2, background activity similar to that of stage 1 is experienced, with bursts of slightly higher frequency “sleep spindles” and sporadic higher amplitude slow wave complexes. The third and fourth stages of sleep display increasing high amplitude slow wave activity. The separate sleep stage in which the individual undergoes rapid eye movement (REM) occupies the remainder of the sleep time and occurs 5 to 6 times during a normal nights sleep. REM sleep is characterized by a lower voltage, higher frequency EEG and other characteristics similar to those which occur when the individual is awake, whereas the other four sleep stages are categorized as NREM sleep.

Individuals vary widely in their requirements for sleep, which is influenced by a number of factors including their current emotional state. The natural aging process is associated with changes in a variety of circadian and diurnal rhythms. Age-related changes in the timing and structure of sleep are surprisingly common problems for older people, and are often associated with significant morbidity. With advancing age, the total amount of sleep tends to shorten. Stage 4 can decrease or disappear and sleep may become more fragmented and interrupted. Evaluation of sleep patterns in elderly people shows that the timing of sleep is also phase advanced, especially in women. This tendency to go to sleep and wake up earlier is very frustrating to older people who feel that they are out of step with the rest of the world. In addition, the quality of sleep in the elderly is diminished with a marked reduction in slow wave sleep, a reduction in the deep stages of sleep (especially stage 4), fragmentation of REM sleep and more frequent awakenings. Similarly, non-elderly people may exhibit disturbances in the normal sleep process. These changes in the structure of sleep have been correlated to more frequent napping, decreased daytime alertness and declining intellectual function and cognitive ability. Deprivation of REM sleep has been suggested to interfere with the memory consolidation involved in learning skills through repetitive activity, and slow wave sleep has been implicated as being important in consolidation of events into long term memory. Likewise, decreases in the length of REM stages of sleep may be associated with a decrease in cognitive function and learning, especially diminished retention of memory. Depression and insomnia may involve a disruption of normal circadian rhythmicity.

Sleep disorders generally involve disturbances of sleep, including circadian rhythm disturbances, that affect a subject's ability to fall and/or stay asleep, and involve sleeping too little, too much or resulting in abnormal behavior associated with sleep.

Numerous compounds are employed in the art to facilitate normal sleep and to treat sleep disorders and sleep disturbances, including e.g., sedatives, hypnotics, anxiolytics, antipsychotics, antianxiety agents, minor tranquilizers, melatonin receptor agonists and antagonists, melatonergic agents, benzodiazepines, barbiturates, 5HT-2 antagonists, and the like. Similarly, physical methods have been employed to treat patients with sleep disorders such as the use of light therapy or the application of modulated electrical signals to selected nerves or nerve bundles.

Nevertheless, the known therapeutic regimens suffer from numerous problems, including residual effects in daytime function, impairment of memory, potential for addiction, rebound insomnia, “REM rebound” which may be associated with increased dream intensity and the occurrence of nightmares, and the like. Accordingly, a more physiological way to enhance sleep, achieve a chronobiologic (circadian rhythm phase-shifting) effect or alleviate circadian rhythm sleep disorders would be highly desirable.

### SUMMARY OF THE INVENTION

The present invention relates to the use of a NPY Y5 antagonist for achieving a chronobiologic (circadian rhythm phase-shifting) effect and alleviating circadian rhythm disorders in a mammal. The present invention is further directed to the use of a NPY Y5 antagonist, for blocking the phase-shifting effects of light in a mammal. Accordingly, the present invention provides a method for achieving a circadian rhythm phase-shifting effect in a mammal comprising the administration of NPY Y5 antagonist. The present invention further provides a pharmaceutical composition for achieving a circadian rhythm phase-shifting effect. The present invention further provides a method of manufacture of a medicament useful for achieving a circadian rhythm phase-shifting effect, for the treatment or prevention of a circadian rhythm disorder, and for blocking the phase-shifting effects of light.

The present invention further relates to the use of a NPY Y5 antagonist for enhancing or improving sleep quality, in particular by increasing sleep efficiency and augmenting sleep maintenance, as well as for preventing and treating sleep disorders and sleep disturbances, in a mammal. Accordingly, the present invention provides a method for enhancing or improving sleep quality and increasing sleep efficiency and sleep maintenance in a mammal comprising the administration of NPY Y5 antagonist. The present invention further provides a pharmaceutical composition for enhancing or improving sleep quality and increasing sleep efficiency and sleep maintenance. The present invention further provides a method of manufacture of a medicament useful for enhancing and improving the quality of sleep, and for the treatment of sleep disorders and sleep disturbances.

### DESCRIPTION OF THE INVENTION

The present invention is directed to the use of NPY Y5 antagonist, for achieving a chronobiologic (circadian rhythm phase-shifting) effect and alleviating circadian rhythm disorders in a mammal. The present invention is further directed to the use of NPY Y5 antagonist, for modulating the phase-shifting effects of light in a mammal.

In an embodiment, the present invention provides a method for the phase advance or phase delay in the circadian rhythm of a subject which comprises administering to the subject an appropriate amount of a NPY Y5 antagonist.

The administration to a subject of an appropriate amount of a NPY Y5 antagonist is useful, for example, in the prevention or treatment of the following conditions to achieve chronobiologic (circadian rhythm phase-shifting) effects and/or to alleviate circadian rhythm phase disorders or disturbances: disorders of the sleep-wake schedule; jet lag; shift work; people who have a maladaption to work and off-work schedules; medical residents, nurses, firemen, policemen or those whose duties require alertness and wakefulness at evening or nighttime hours, or those deprived of sleep for various periods because of their duties or responsibilities; animal workers; athletes who wish to reset their internal clock to a more beneficial time; the infantry, or other members of the armed forces whose duties require extreme levels of alertness and wakefulness, and those who may be sleep deprived in the performance of these duties; submariners, or people confined for research, exploration or industrial purposes below the seas; miners, spelunkers, researchers or those confined beneath the Earth; astronauts in orbit around the Earth, on missions in space to the Earth's moon or to the planets or out of the solar system, or in training for such missions; the blind or sight-impaired or those persons whose ability to distinguish differences in light and dark may be permanently or temporarily impaired; psychiatric patients; those with night eating syndrome, insomniacs; the comatose, or those who need to be maintained in a state of unconsciousness for medical, psychiatric or other reasons; residents of the far North or Antarctica, or those persons who live in a climate or climates which possess abnormal amounts of light or darkness; those suffering from seasonal affective disorder (SAD), winter depression, or other forms of depression; the aged; Alzheimer's disease patients, or those suffering from other forms of dementia; patients who require dosages of medication at appropriate times in the circadian cycles; patients suffering from delayed sleep phase syndrome, advanced sleep phase syndrome, or non-24 hr sleep phase syndrome; and patients suffering from primary or secondary insomnia or circadian rhythm-related insomnia.

Circadian rhythms affect a variety of physiological parameters: rest-activity, sleep-wake cycles, body temperature, rhythms in hormone levels, oscillations in general physiology and the like. When these parameters are out of synchrony with the daily clock, a circadian rhythm imbalance occurs which can affect physiology, performance on a variety of tasks and one's emotional well being. The present invention is useful, for example, in the prevention or treatment of conditions associated with circadian rhythmicity as well as mental and physical disorders associated with travel across time zones and with rotating shift-work schedules.

In another embodiment, the present invention provides a method for the prevention or treatment of a circadian rhythm disorder in a mammal, including time-zone change (jet-lag) syndrome, shift-work sleep disorder, delayed sleep-phase syndrome, advanced sleep-phase syndrome, and non-24-hour sleep-wake disorder, which comprises administering to the mammal an effective amount of a NPY Y5 receptor antagonist.

In another embodiment, the present invention provides a method for shortening the time of re-entrainment (return to normal entrainment of the circadian rhythms; synchronized to the environmental light-dark cycle) in a subject following a shift in the sleep-wake cycle which comprises administering to the subject an appropriate amount of a NPY Y5 antagonist.

In another embodiment, the present invention provides a method for alleviating the effects of jet lag in a traveler, especially a mammal, which comprises administering to the traveler an alertness increasing amount of a NPY Y5 antagonist. The purpose of this embodiment is to assist the body to adjust physiologically to the changes in sleep and feeding patterns when crossing several time zones.

In another more preferred embodiment, the present invention provides a method for resetting the internal circadian clock in a subject to match the subject's current activity/sleep cycle. For example shift workers changing from a day to a night shift or vice versa, which comprises administering to the subject an appropriate amount of a NPY Y5 antagonist.

The present invention is further directed to the use of NPY Y5 antagonist, for enhancing or improving sleep quality as well as preventing and treating sleep disorders and sleep disturbances in a mammal. In particular, the present invention provides a method for enhancing or improving sleep quality by increasing sleep efficiency and augmenting sleep maintenance. In addition, the present invention provides a method for preventing and treating sleep disorders and sleep disturbances in a mammal which comprising the administration of a NPY Y5 antagonist. The present invention further provides a pharmaceutical composition for enhancing or improving sleep quality and increasing sleep efficiency and sleep maintenance. The present invention is useful for the treatment of sleep disorders, including Disorders of Initiating and Maintaining Sleep (insomnias) (“DIMS”) which can arise from psychophysiological causes, as a consequence of psychiatric disorders (particularly related to anxiety), from drugs and alcohol use and abuse (particularly during withdrawal stages), childhood onset DIMS, nocturnal myoclonus and restless legs and non specific REM disturbances as seen in ageing.

The following outcomes in a subject which are provided by the present invention may be correlated to enhancement in sleep quality: an increase in the value which is calculated from the time that a subject sleeps divided by the time that a subject is attempting to sleep; a decrease in sleep latency (the time it takes to fall asleep); a decrease in the number of awakenings during sleep; a decrease in the time spent awake following the initial onset of sleep; an increase in the total amount of sleep; an increase the amount and percentage of REM sleep; an increase in the duration and occurrence of REM sleep; a reduction in the fragmentation of REM sleep; an increase in the amount and percentage of slow-wave (i.e. stage 3 or 4) sleep; an increase in the amount and percentage of stage 2 sleep; a decrease in the number of awakenings, especially in the early morning; an increase in daytime alertness; and increased sleep maintenance. Secondary outcomes which may be provided by the present invention include enhanced cognitive function and increased memory retention. A “method for enhancing the quality of sleep” refers to a method that results in outcomes in a subject which may be correlated to enhancement in sleep quality, including, but not limited to, the outcomes correlated to enhancement of sleep quality as defined above.

The present invention is further useful for the prevention and treatment of sleep disorders and sleep disturbances including sleep problems associated with insomnia, hypersomnia, sleep apnea, narcolepsy, nocturnal myoclonus, REM sleep interruptions, jet-lag, shift workers' sleep disturbances, dysomnias, night terror, night eating syndrome, insomnias associated with depression or with emotional/mood disorders, dysfunctions associated with sleep (parasomnias), as well as sleep walking and enuresis, as well as sleep disorders which accompany aging. Sleep disorders and sleep disturbances are generally characterized by difficulty in initiating or maintaining sleep or in obtaining restful or enough sleep.

In addition, certain drugs may also cause reductions in REM sleep as a side effect and the present invention may be used to correct those types of sleeping disorders as well. The present invention would also be of benefit in the treatment of syndromes such as fibromyalgia which are manifested by non-restorative sleep and muscle pain or sleep apnea which is associated with respiratory disturbances during sleep. It will be clear to one skilled in the art that the present invention is not limited to just sleep disorders and sleep disturbances, but is applicable to a wide variety of conditions which result from a diminished quality of sleep.

The present invention is also concerned with treatment and prevention of these conditions, and with the use of a NPY Y5 antagonist, combinations, and compositions thereof, for the manufacture of a medicament useful for treating or preventing these conditions.

In the present invention, it is preferred that the subject mammal is a human. Although the present invention is applicable both old and young people, it may find greater application in elderly people. Further, although the invention may be employed to enhance the sleep of healthy people, it may be especially beneficial for enhancing the sleep quality of people suffering from sleep disorders or sleep disturbances.

The NPY Y5 antagonists of use in the present invention may be any NPY Y5 antagonist known from the art.

The NPY Y5 antagonist may be peptidal or non-peptidal in nature, however, the use of a non-peptidal NPY Y5 antagonist is preferred. In addition, for convenience the use of an orally active NPY Y5 antagonist is preferred.

In the present invention, it is preferred that the NPY Y5 antagonist active upon the central nervous system (CNS), such as the brain, following systemic administration, i.e. that it readily penetrates the CNS. Accordingly, a preferred NPY Y5 antagonist for use in the present invention is a CNS-penetrating NPY Y5 antagonist.

Non-limiting examples of Y5 receptor antagonists include compounds of the formula: 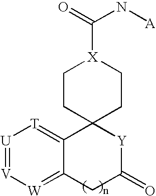
wherein A is selected from the group consisting of aryl or heteroaryl, wherein said aryl and heteroaryl groups may be optionally substituted on either the carbon or hetero atom, the substituent being selected from the group consisting of halogen, nitro, lower alkyl, halo(lower)alkyl, hydroxy(lower)alkyl, cyclo(lower)alkyl, lower alkenyl, lower alkoxy, halo(lower)alkoxy, lower alkylthio, carboxyl, lower alkanoyl, lower alkoxycarbonyl, lower alkylene optionally substituted with oxo, and a group represented by formula of -Q-D;

D is selected from the group consisting of aryl or heteroaryl, wherein said aryl and heteroaryl groups may be optionally substituted, the substituent being selected from the group consisting of halogen, cyano, lower alkyl, halo(lower)alkyl, hydroxy(lower)alkyl, hydroxy, lower alkoxy, halo(lower)alkoxy, lower alkylamino, di-lower alkylamino, lower alkanoyl and aryl;

n is an integer from 0 to 1;

Q is selected from the group consisting of a single bond or carbonyl;

T, U, V and W are each independently selected from the group consisting of nitrogen or a methylene group, said nitrogen or methylene group may be optionally substituted with a substituent selected from the group consisting of: halogen, lower alkyl, hydroxy, and lower alkoxy;

X is selected from the group consisting of methylene or nitrogen;

Y is selected from the group consisting of nitrogen and oxygen, wherein said nitrogen may be optionally substituted with lower alkyl or oxygen;

and the pharmaceutically acceptable salts and esters thereof. These compounds are further described and methods of preparing them can be found in International Publication Number WO 01/14376, and in U.S. Pat. Nos. 6,326,375, and 6,335,345, which are hereby incorporated by reference in their entirety.

Non-limiting examples of NPY Y5 receptor antagonists include compounds of the formula: 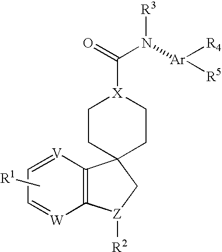
or a pharmaceutically acceptable salt thereof, wherein;

V, W, X and Z are independently selected from CH and N;

R¹ is H, C₁₋₃ alkyl, C₁₋₃ alkoxy, F, or Cl;

R² is S(O)n R⁶, COR⁶ or CHO, wherein

n is 0, 1 or 2; and

R⁶ is N(R³)₂ or C₁₋₃ alkyl;

R³ is independently H or C₁₋₃ alkyl;

Ar is aryl or heteroaryl;

R⁴ and R⁵ are independently selected from:
(1) hydrogen,
(2) aryl, either unsubstituted or substituted with
   (a) halo
   (b) C₁₋₃ alkoxy,
   (c) —N(C₁₋₃ alkyl)₂,
   (d) C₂₋₄ alkanoyl, or
   (e) aryl;
(3) nitro,
(4) C₁₋₅ alkyl,
(5) C₁₋₅ alkoxy,
(6) hydroxy-C₁₋₃ alkyl,
(7) carboxy,
(8) halo,
(9) C₁₋₅ alkylthio,
(10) C₁₋₅ ethoxycarbonyl,
(11) pyridylcarbonyl,
(12) benzoyl,
(13) phenyl-C₁₋₃ alkoxy,
(14) pyridyl, either unsubstituted or substituted with C₁₃ alkyl or C₁₋₃ alkoxy,
(15) C₃₋₆ cycloalkyl,
(16) oxazolyl,
(17) thiazolyl,
(18) triazolyl,
(19) phenoxy, and
(20) C₂₋₆ alkanoyl.

These compounds are further described and methods of preparing them can be found in International Publication Number WO 00/27845, and U.S. Pa. Nos. 6,191,160, and 6,313,298, which is hereby incorporated by reference in their entirety.

Non-limiting examples of NPY Y5 receptor antagonists include compound L-152,804 of the formula: 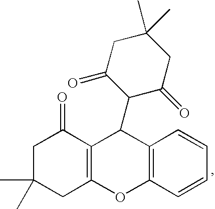
and pharmaceutically acceptable salts, esters and tautomers thereof. Compound L-152,804 and its preparation are disclosed in J. Organic Chemistry, vol. 31, No. 5, p. 1639 (1966); and U.S. Pat. No. 6,258,837, which is hereby incorporated by reference in its entirety.

The above compounds are only illustrative of the NPY Y5 antagonists which are currently under investigation. As this listing of groups of compounds is not meant to be comprehensive, the methods of the present invention may employ any NPY Y5 antagonist and is not limited to any particular structural class of compound.

Suitable pharmaceutically acceptable salts of the NPY Y5 antagonists of use in the present invention include acid addition salts which may, for example, be formed by mixing a solution of the compound with a solution of a pharmaceutically acceptable non-toxic acid such as hydrochloric acid, fumaric acid, maleic acid, succinic acid, acetic acid, citric acid, tartaric acid, carbonic acid, phosphoric acid or sulphuric acid. Salts of amine groups may also comprise the quaternary ammonium salts in which the amino nitrogen atom carries an alkyl, alkenyl, alkynyl or aralkyl group. Where the compound carries an acidic group, for example a carboxylic acid group, the present invention also contemplates salts thereof, preferably non-toxic pharmaceutically acceptable salts thereof, such as the sodium, potassium and calcium salts thereof.

Certain of the above defined terms may occur more than once in the above formula and upon such occurrence each term shall be defined independently of the other. Similarly, the use of a particular variable within a noted structural formula is intended to be independent of the use of such variable within a different structural formula.

Full descriptions of the preparation of the NPY Y5 antagonists which are employed in the present invention may be found in the references cited herein.

The identification of a compound as a NPY Y5 antagonist, in particular a CNS penetrant NPY Y5 antagonist, and thus able to have utility in the present invention may be readily determined without undue experimentation by methodology well known in the art, such as the assays described herein.

The NPY Y5 antagonist may be used alone or in conjunction with other agents which are known to be beneficial in altering circadian rhythms or in the enhancement of sleep efficiency. The NPY Y5 antagonist and the other agent may be co-administered, either in concomitant therapy or in a fixed combination, or they may be administered at separate times. For example, the NPY Y5 antagonist may be administered in conjunction with other compounds which are known in the art to be useful for suppressing or stimulating melatonin production including melatonergic agents, noradrenergic and serotonergic re-uptake blockers, alpha-1-noradrenergic agonists, monamine oxidase inhibitors, other NPY agonists or antagonists; neurokinin-1 agonists; substance P; beta-adrenergic blockers and benzodiazepines, such as atenolol; or with other compounds which are known in the art to be useful for stimulating melatonin production including tricyclic antidepressants and alpha-2-adrenergic antagonists; or with melatonin precursors such as tryptophan, 5-hydroxytryptophan, serotonin and N-acetylserotonin; as well as melatonin analogs, melatonin agonists and melatonin antagonists, or melatonin itself.

In addition, the NPY Y5 antagonist may be administered in conjunction with other compounds which are known in the art to be useful for enhancing sleep quality and preventing and treating sleep disorders and sleep disturbances, including e.g., sedatives, hypnotics, anxiolytics, antipsychotics, antianxiety agents, minor tranquilizers, melatonin agonists and antagonists, melatonin, melatonergic agents, benzodiazepines, barbiturates, 5HT-2 antagonists, and the like, such as: adinazolam, allobarbital, alonimid, alprazolam, amitriptyline, amobarbital, amoxapine, bentazepam, benzoctamine, brotizolam, bupropion, busprione, butabarbital, butalbital, capuride, carbocloral, chloral betaine, chloral hydrate, chlordiazepoxide, clomipramine, cloperidone, clorazepate, clorethate, clozapine, cyprazepam, desipramine, dexclamol, diazepam, dichloralphenazone, divalproex, diphenhydramine, doxepin, estazolam, ethchlorvynol, etomidate, fenobam, flunitrazepam, flurazepam, fluvoxamine, fluoxetine, fosazepam, glutethimide, halazepam, hydroxyzine, imipramine, lithium, lorazepam, lormetazepam, maprotiline, mecloqualone, melatonin, mephobarbital, meprobamate, methaqualone, midaflur, midazolam, nefazodone, nisobamate, nitrazepam, nortriptyline, oxazepam, paraldehyde, paroxetine, pentobarbital, perlapine, perphenazine, phenelzine, phenobarbital, prazepam, promethazine, propofol, protriptyline, quazepam, reclazepam, roletamide, secobarbital, sertraline, suproclone, temazepam, thioridazine, tracazolate, tranylcypromaine, trazodone, triazolam, trepipam, tricetamide, triclofos, trifluoperazine, trimetozine, trimipramine, uldazepam, valproate, venlafaxine, zaleplon, zolazepam, zolpidem, and salts thereof, and combinations thereof, and the like.

The NPY Y5 antagonist may be administered in conjunction with the use of physical methods such as with light therapy or electrical stimulation. In particular, the NPY Y5 antagonist may be administered in conjunction with scheduling bright light administration, ordinary-intensity light exposure, or exposure to dim-light or darkness (or even sleep). In one embodiment of the present invention, the NPY Y5 antagonist is administered accompanied by having an individual wear dark or red goggles at the time of administration to provide additive effects of the treatment plus darkness. In another embodiment of the present invention, the individual wears dark goggles at times other than the time of NPY Y5 antagonist administration to avoid the occurrence of an external zeitgeber with respect to the phase shift resulting from the NPY Y5 antagonist. Similarly, bright light exposure can be used in conjunction with administration of a NPY Y5 antagonist. As used herein, the term “light therapy” includes, but is not limited to, the above definitions of light therapy.

Accordingly, the present invention further includes within its scope the use of a NPY Y5 antagonist, alone or in combination with other agents, for altering circadian rhythms or for the prevention or treatment of sleep disorders and sleep disturbances in a mammal. The preferred mammal for purposes of this invention is human.

It will be appreciated to those skilled in the art that reference herein to treatment extends to prophylaxis (prevention) as well as the treatment of the noted diseases/disorders and symptoms.

Included within the scope of the present invention is the method of using a NPY Y5 antagonist for altering circadian rhythms or for enhancing and improving the quality of sleep. The NPY Y5 antagonist is useful in enhancing or improving sleep quality as well as preventing and treating sleep disorders and sleep disturbances in a mammal. In addition, the use of the NPY Y5 antagonist increases sleep efficiency and augments sleep maintenance. The NPY Y5 antagonist may further be used in a method for preventing and treating sleep disorders and sleep disturbances in a mammal. The present invention further provides a pharmaceutical composition for altering circadian rhythms or for enhancing or improving sleep quality and increasing sleep efficiency and sleep maintenance.

The present method of using a NPY Y5 antagonist further provides the following: an increase in the value which is calculated from the time that a subject sleeps divided by the time that a subject is attempting to sleep; a decrease in sleep latency (the time it takes to fall asleep); a decrease in the number of awakenings during sleep; a decrease in the time spent awake following the initial onset of sleep; an increase in the total amount of sleep; an increase the amount and percentage of REM sleep; an increase in the duration and occurrence of REM sleep; a reduction in the fragmentation of REM sleep; an increase in the amount and percentage of slow-wave (i.e. stage 3 or 4) sleep; an increase in the amount and percentage of stage 2 sleep; a decrease in the number of awakenings, especially in the early morning; an increase in daytime alertness; and increased sleep maintenance; enhanced cognitive function; and increased memory retention.

The present invention is further useful for the prevention and treatment of sleep disorders and sleep disturbances including: sleep problems associated with insomnia, hypersomnia, sleep apnea, narcolepsy, nocturnal myoclonus, REM sleep interruptions, jet-lag, shift workers' sleep disturbances, dysomnias, night terror, insomnias associated with depression or with emotional/mood disorders, as well as sleep walking and enuresis, as well as sleep disorders which accompany aging, conditions associated with circadian rhythmicity, mental and physical disorders associated with travel across time zones and with rotating shift-work schedules, or syndromes such as fibromyalgia which are manifested by non-restorative sleep and muscle pain or sleep apnea which is associated with respiratory disturbances during sleep.

In addition, the present invention includes within its scope a pharmaceutical composition for enhancing and improving the quality of sleep comprising, as an active ingredient, at least one NPY Y5 antagonist in association with a pharmaceutical carrier or diluent. The present invention further includes the use of a NPY Y5 antagonist in the manufacture of a medicament for achieving a circadian rhythm phase-shifting effect, alleviating a circadian rhythm disorder, blocking the phase-shifting effects of light, enhancing and improving the quality of sleep, or for the treatment of sleep disorders or sleep disturbances.

It will be known to those skilled in the art that there are numerous compounds now being used to affect circadian rhythms or to enhance and improve the quality of sleep. Combinations of these therapeutic agents some of which have also been mentioned herein with a NPY Y5 antagonist will bring additional, complementary, and often synergistic properties to enhance the desirable properties of these various therapeutic agents. In these combinations, the NPY Y5 antagonist and the therapeutic agents may be independently present in dose ranges from one one-hundredth to one times the dose levels which are effective when these compounds are used singly.

The NPY Y5 antagonist may be administered in combination with sedatives, hypnotics, anxiolytics, antipsychotics, antianxiety agents, minor tranquilizers, melatonin agonists and antagonists, melatonergic agents, benzodiazepines, barbiturates, 5HT-2 antagonists, and the like, or the NPY Y5 antagonist may be administered in conjunction with the use of physical methods such as with light therapy or electrical stimulation. For example, to alter circadian rhythmicity or to enhance and improve the quality of sleep a NPY Y5 antagonist may be given in combination with such compounds as: adinazolam, allobarbital, alonimid, alprazolam, amitriptyline, amobarbital, amoxapine, bentazepam, benzoctamine, brotizolam, bupropion, busprione, butabarbital, butalbital, capuride, carbocloral, chloral betaine, chloral hydrate, chlordiazepoxide, clomipramine, cloperidone, clorazepate, clorethate, clozapine, cyprazepam, desipramine, dexclamol, diazepam, dichloralphenazone, divalproex, diphenhydramine, doxepin, estazolam, ethchlorvynol, etomidate, fenobam, flunitrazepam, flurazepam, fluvoxamine, fluoxetine, fosazepam, glutethimide, halazepam, hydroxyzine, imipramine, lithium, lorazepam, lormetazepam, maprotiline, mecloqualone, melatonin, mephobarbital, meprobamate, methaqualone, midaflur, midazolam, nefazodone, nisobamate, nitrazepam, nortriptyline, oxazepam, paraldehyde, paroxetine, pentobarbital, perlapine, perphenazine, phenelzine, phenobarbital, prazepam, promethazine, propofol, protriptyline, quazepam, reclazepam, roletamide, secobarbital, sertraline, suproclone, temazepam, thioridazine, tracazolate, tranylcypromaine, trazodone, triazolam, trepipam, tricetamide, triclofos, trifluoperazine, trimetozine, trimipramine, uldazepam, valproate, venlafaxine, zaleplon, zolazepam, zolpidem, and salts thereof, and combinations thereof, and the like, as well as admixtures and combinations thereof.

Typically, the individual daily dosages for these combinations may range from about one-fifth of the minimally recommended clinical dosages to the maximum recommended levels for the entities when they are given singly.

To illustrate these combinations, a NPY Y5 antagonist effective clinically at a given daily dose range may be effectively combined, at levels which are equal or less than the daily dose range, with the following compounds at the indicated per day dose range: adinazolam, allobarbital, alonimid, alprazolam, amitriptyline, amobarbital, amoxapine, bentazepam, benzoctamine, brotizolam, bupropion, busprione, butabarbital, butalbital, capuride, carbocloral, chloral betaine, chloral hydrate, chlordiazepoxide, clomipramine, cloperidone, clorazepate, clorethate, clozapine, cyprazepam, desipramine, dexclamol, diazepam, dichloralphenazone, divalproex, diphenhydramine, doxepin, estazolam, ethchlorvynol, etomidate, fenobam, flunitrazepam, flurazepam, fluvoxamine, fluoxetine, fosazepam, glutethimide, halazepam, hydroxyzine, imipramine, lithium, lorazepam, lormetazepam, maprotiline, mecloqualone, melatonin, mephobarbital, meprobamate, methaqualone, midaflur, midazolam, nefazodone, nisobamate, nitrazepam, nortriptyline, oxazepam, paraldehyde, paroxetine, pentobarbital, perlapine, perphenazine, phenelzine, phenobarbital, prazepam, promethazine, propofol, protriptyline, quazepam, reclazepam, roletamide, secobarbital, sertraline, suproclone, temazepam, thioridazine, tracazolate, tranylcypromaine, trazodone, triazolam, trepipam, tricetamide, triclofos, trifluoperazine, trimetozine, trimipramine, uldazepam, venlafaxine, zaleplon, zolazepam, zolpidem, and salts thereof, and combinations thereof, and the like, as well as admixtures and combinations thereof. It will be readily apparent to one skilled in the art that the NPY Y5 antagonist may be employed with other agents to alter circadian rhythms or to control sleep disorders and sleep disturbances in depressed patients and/or provide benefit in the prevention or treatment of sleep disorders and sleep disturbances.

Naturally, these dose ranges may be adjusted on a unit basis as necessary to permit divided daily dosage and, as noted above, the dose will vary depending on the nature and severity of the disease, weight of patient, special diets and other factors.

These combinations may be formulated into pharmaceutical compositions as known in the art and as discussed below. A NPY Y5 antagonist may be administered alone or in combination by oral, parenteral (e.g., intramuscular, intraperitoneal, intravenous or subcutaneous injection, or implant), nasal, vaginal, rectal, sublingual, or topical routes of administration and can be formulated in dosage forms appropriate for each route of administration.

Solid dosage forms for oral administration include capsules, tablets, pills, powders and granules. In such solid dosage forms, the active compound is admixed with at least one inert pharmaceutically acceptable carrier such as sucrose, lactose, or starch. Such dosage forms can also comprise, as is normal practice, additional substances other than inert diluents, e.g., lubricating agents such as magnesium stearate. Illustrative of the adjuvants which may be incorporated in tablets, capsules and the like are the following: a binder such as gum tragacanth, acacia, corn starch or gelatin; an excipient such as microcrystalline cellulose; a disintegrating agent such as corn starch, pregelatinized starch, alginic acid and the like; a lubricant such as magnesium stearate; a sweetening agent such as sucrose, lactose or saccharin; a flavoring agent such as peppermint, oil of wintergreen or cherry. In the case of capsules, tablets and pills, the dosage forms may also comprise buffering agents.

When the dosage unit form is a capsule, it may contain, in addition to materials of the above type, a liquid carrier such as fatty oil. Various other materials may be present as coatings or to otherwise modify the physical form of the dosage unit. Tablets and pills can additionally be prepared with enteric coatings and tablets may be coated with shellac, sugar or both.

Liquid dosage forms for oral administration include pharmaceutically acceptable emulsions, solutions, suspensions, syrups, the elixirs containing inert diluents commonly used in the art, such as water. Besides such inert diluents, compositions can also include adjuvants, such as wetting agents, emulsifying and suspending agents, and sweetening, flavoring, and perfuming agents. A syrup or elixir may contain the active compound, sucrose as a sweetening agent, methyl and propyl parabens as preservatives, a dye and a flavoring such as cherry or orange flavor.

Preparations according to this invention for parenteral administration include sterile aqueous or non-aqueous solutions, suspensions, or emulsions. Sterile compositions for injection may be formulated according to conventional pharmaceutical practice by dissolving or suspending the active substance in a vehicle such as water for injection, a naturally occurring vegetable oil like sesame oil, coconut oil, peanut oil, cottonseed oil, etc., or a synthetic fatty vehicle like ethyl oleate or the like. Buffers, preservatives, antioxidants and the like may be incorporated as required. Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized by, for example, filtration through a bacteria-retaining filter, by incorporating sterilizing agents into the compositions, by irradiating the compositions, or by heating the compositions. They can also be manufactured in the form of sterile solid compositions which can be dissolved in sterile water, or some other sterile injectable medium immediately before use. Compositions for rectal or vaginal administration are preferably suppositories which may contain, in addition to the active substance, excipients such as cocoa butter or a suppository wax. Compositions for nasal or sublingual administration are also prepared with standard excipients well known in the art.

The dosage of active ingredient in the compositions of this invention may be varied, however, it is necessary that the amount of the active ingredient be such that a suitable dosage form is obtained. The active ingredient may be administered to patients (animals and human) in need of such treatment in dosages that will provide optimal pharmaceutical efficacy. The selected dosage depends upon the desired therapeutic effect, on the route of administration, and on the duration of the treatment. As will be readily apparent to one skilled in the art, the effect of a NPY Y5 antagonist which induces a phase shift in a central circadian pacemaker may be dependent on both the ambient and circadian time of administration. The same compound may induce a phase advance, a phase delay or have minor effect on a particular circadian rhythm depending on the circadian time of administration. The dose will vary from patient to patient depending upon the nature and severity of disease, the patient's weight, special diets then being followed by a patient, concurrent medication, the intrinsic NPY Y5 antagonist activity of the compound, the bioavailability upon oral administration of the compound and other factors which those skilled in the art will recognize.

In the treatment of a condition in accordance with the present invention, an appropriate dosage level, will generally be about 0.01 μg to 50 mg per kg patient body weight per day which may be administered in single or multiple-doses. Preferably, the dosage level will be about 0.1 μg to about 25 mg/kg per day; more preferably about 0.5 μg to about 10 mg/kg per day. For example, for achieving a circadian rhythm phase-shifting effect, resetting the internal circadian clock, shortening the time of re-entrainment of circadian rhythms, alleviating a circadian rhythm disorder, increasing alertness, or enhancing the quality of sleep, a suitable dosage level, is about 0.1 μg to 25 mg/kg per day, preferably about 0.5 μg to 10 mg/kg per day, and especially about 1 μg to 5 mg/kg per day. In larger mammals, for example humans, a typical indicated dose is about 300 μg to 400 mg orally. A compound may be administered on a regimen of several times per day, for example 1 to 4 times per day, preferably once or twice per day. When using an injectable formulation, a suitable dosage level is about 0.1 μg to 10 mg/kg per day, preferably about 0.5 μg to 5 mg/kg per day, and especially about 1 μg to 1 mg/kg per day. In larger mammals, for example humans, a typical indicated dose is about 100 μg to 100 mg i.v. A compound may be administered on a regimen of several times per day, for example 1 to 4 times per day, preferably once or twice per day.

Pharmaceutical compositions of the present invention may be provided in a solid dosage formulation preferably comprising about 100 μg to 500 mg active ingredient, more preferably comprising about 100 μg to 250 mg active ingredient. The pharmaceutical composition is preferably provided in a solid dosage formulation comprising about 100 μg, 1 mg, 5 mg, 10 mg, 25 mg, 50 mg, 100 mg, 200 mg or 250 mg active ingredient.

The following examples are provided for the purpose of further illustration only and are not intended to be limitations on the disclosed invention.

### EXAMPLE 1

Y5 receptor antagonist assay:

To identify a potent Y5 antagonist for treatment of circadian rhythm disorders in humans, the cloned human Y5 receptor is used in the primary assay. Vectors expressing either the 455 amino acid form (See, e.g., U.S. Pat. No 5,602,024) or a 10 amino acid, N-terminally shorter form (See, e.g., U.S. Pat. No. 5,919,901) can be introduced into cell lines to obtain cells which express the human Y5 receptor. Binding of [¹²⁵I]PYY (NEN) to membrane preparations from cells expressing the cloned human Y5 receptor are performed in 0.2 ml of 25 mM Tris buffer (pH 7.4) containing 10 mM MgCl₂, 1 mM PMSF, 0.1% bacitracin and 0.5% bovine serum albumin. Membranes (10-300 μg/ml) prepared from LMtk-, COS-7, HEK or CHO cells expressing Y5 receptors, are incubated at 25° C. for 120 min with [¹²⁵I]PYY (25 pM) in the presence of several concentrations of compounds to be evaluated. Bound and free peptides are separated by filtration using a GF/C glass filter presoaked with 0.3% polyethylenimine. The remaining radioactivity on the filter is quantitated using a TopCount™ (Packard Instruments Co. Inc.). Specific binding of [¹²⁵I]PYY is defined as the difference between total binding and nonspecific binding in the presence of 1 μM PYY. The binding IC₅₀ is calculated using GraphPad Prism (Ver. 3.0).

The functional potency of Y5 antagonists can be determined using various assays which measure inhibition of second messenger pathways. NPY increases intracellular Ca²⁺ concentration via activation of Y5 receptors through coupling to Gαqi5. The potency of a Y5 antagonist in blocking NPY mediated Ca²⁺ increase can be used as a measure of its functional antagonist activity. For example, CHO cells expressing both NPY Y5 receptors and Gαqi5 are seeded (40,000 cells per well) into 96-well plate 24 hr before assay. Cells are loaded for 1 hr with a Ca²⁺-sensitive fluorescent dye, Fluo-4-AM in assay buffer (Hank's Balanced Salts Solution (HBSS) containing 20 mM HEPES, 0.5 % BSA and 2.5 mM probenecid, pH 7.4), washed 3 times with the assay buffer, then returned to the incubator for 1 hr before assay on a fluorometric imaging plate reader, FLIPR™ (Molecular Device, California). The NPY-induced maximum change in fluorescence over baseline is determined and the dose which induces a 50% increase in fluorescence is defined as the EC₅₀ dose for NPY. To evaluate Y5 antagonists, the assay is repeated with the EC₅₀ dose of NPY in the presence of various concentrations of a Y5 antagonist to generate a functional IC₅₀. The concentration-response curves are fitted using GraphPad Prism (Ver. 3.0). Using these assays, potent Y5 antagonists with a binding IC₅₀ and/or functional IC₅₀ of less than 1 μM can be identified. Useful antagonists would also have to posses other characteristics such as selectivity over the other NPY receptors, good systemic exposure, sufficient half-life and brain penetration.

### EXAMPLE 2

Assessment of behavioral rhythms:

For the recording of locomotor activity rhythms in rodents, animals would be maintained under a standard light-dark cycle (12:12 or 14:10, depending upon the species or strain of rodent) for several weeks prior to the start of each experiment. Animals would be housed individually with access to a running wheel in the cage and wheel-running activity would be recorded continuously (e.g., using a Chronobiology kit, Actiview software, or another biological rhythm analysis software package). Food (rodent chow) and water would be available ad libitum.

(1) To evaluate whether NPY5 receptor antagonist treatment phase-shifts the circadian rhythm of locomotor activity, one would administer the NPY5 receptor antagonist or vehicle to animals at different circadian times while the animals were maintained under dim red light conditions (<15 lux of light) in “constant dark” conditions. Animals would be dosed with the NPY5 receptor antagonist at times when one would expect to observe phase delays (e.g., early subjective night, circadian time (CT) 14) and phase advances (e.g., late subjective night, CT 19) in wheel-running activity to generate a phase-response curve.

(2) To evaluate the effects of NPY5 antagonists on light-induced phase shifts of a rodent circadian locomotor activity rhythm, animals would be housed in constant darkness and then injected with the NPY5 antagonist or vehicle 30 minutes prior to exposure to a light-pulse at a behaviorally relevant time (i.e., at a time when light would produce large phase advances or phase delays in behavior as outlined above; CT14 and/or CT19). For light stimulation, the animals would be exposed to ˜100-300 lux of fluorescent white light for 10-15 minutes. The locomotor activity of the animals would be monitored for several days following the light pulse.

(3) To test whether injections of NPY5 antagonists mimic the effect of dark pulses in animals housed in constant light, animals would be kept in constant light (˜100-300 lux). Dark pulses induce phase advances and phase delays in locomotor activity rhythms when applied, respectively, during the mid-subjective day and the late subjective night. A subset of animals would be treated with NPY5 antagonists or vehicle during the mid-subjective day (e.g., CT6 or CT8) and another group of animals would be similarly treated with NPY antagonists or vehicle during the late subjective night (e.g., CT19).

(4) To evaluate whether daily NPY5 receptor antagonist treatment would entrain free-running locomotor activity rhythms in rodents maintained in constant darkness (under dim red safelight;15 W, Kodak 1A filter), animals would be maintained in constant darkness for several days or weeks to stabilize their free-running activity rhythms. Animals would be dosed with an NPY5 receptor antagonist or vehicle for several consecutive days (e.g, ranging from 3-21 days) at the same clock time (e.g., CT10 or CT24). In this paradigm, the onset of wheel-running activity would “lock onto” the time at which the NPY5 receptor antagonist is administered. Entrainment would be defined as when the onset of locomotor activity coincided with the time of daily injections.

To determine phase-shifts in wheel-running rhythms for these studies, actograms (plots of activity data) would be examined and regression curves would be fitted by eye (or with the software) to the onsets of locomotor activity for 7-10 days prior to the drug treatment and projected to the day of treatment. This method would be used to extrapolate the magnitude of the phase-shift (i.e, the difference between the lines).

### EXAMPLE 3

Determine the effect of a Y5 antagonist, L-152804 on phase shifts of the circadian rhythm

A. Effect of L-152804 on circadian rhythms

Male golden hamsters are housed individually under a Light-Dark (LD) cycle of 14:10 upon arrival from the supplier for 2 weeks. All animals are housed with a running wheel to measure activity. The animals are then transferred to a constant dark (DD) cycle for 2 weeks and activity records from the running wheel are used to determine circadian time (CT) 14.

Two groups of hamsters are treated orally with vehicle (0.5% methocel) or L-152804 at 50 mg/kg, then the animals are returned to their home cages and activity recorded for 10 days by monitoring the time the animals spend on a running wheel. A shift in the peak time of activity would indicate a Y5 antagonist can shift the circadian rhythm.

B. Effect of L-152804 on light induced phase shifts in the circadian rhythm

Male golden hamsters are housed individually under a Light-Dark (LD) cycle of 14:10 upon arrival from the supplier for 2 weeks. All animals are housed with a running wheel to measure activity. The animals are then transferred to a constant dark (DD) cycle for 2 weeks and activity records from the running wheel are used to determine circadian time (CT) 14.

Two groups of hamsters are treated orally with vehicle (0.5% methocel) or L-152804 at 50 mg/kg, then subjected to light pulses at two different behaviorally-relevant times: CT14 and CT 20. Light pulses (503 nm, 8.6×10¹² photons/cm2/sec, 5 min), which are known to induce a phase shift in the activity level of hamsters of about ⅓ the maximal shift, are given to both groups. The animals are returned to their home cages and activity recorded for 10 days. The Y5 antagonist treatment enhances the effect of the light pulse if the phase shift is greater then the effect of vehicle treatment. Alternatively, the Y5 antagonist inhibits the light-induced phase shift if the phase shift is less than that seen with the vehicle treated animals.

C. Effect of L-152804 activity induced phase shifts in the circadian rhythm

Male golden hamsters are housed individually under a Light-Dark (LD) cycle of 14:10 upon arrival from the supplier for 2 weeks. All animals are housed with a running wheel to measure activity. The animals are then transferred to a constant dark (DD) cycle for 2 weeks and activity records from the running wheel are used to determine circadian time (CT) 8.

Two groups of hamsters are treated orally with vehicle (0.5% methocel) or L-152804 at 50 mg/kg, then subjected to a single injection of the benzodiazepene agonist triazolam (3 mg/kg) at CT8. The animals are returned to their home cages and activity recorded for 10 days to determine if L-152804 suppresses the circadian shifts induced by the activity stimulus, triazolam.

### EXAMPLE 4

Double-Blind, Placebo-Controlled Study to Determine the Effect of a Neuropeptide Y Y5 antagonist on Light-Induced Melatonin Suppression in Healthy Young Men

The purpose of this study is to evaluate the effects of a neuropeptide Y Y5 antagonist on circadian rhythms in humans by examining the amount of light-induced melatonin suppression in subjects treated with placebo or the neuropeptide Y Y5 antagonist. If a neuropeptide Y Y5 antagonist is able to alter the amount of light-induced melatonin suppression and so influence circadian rhythms, it may be a useful agent, e.g., for treating jet lag, shift workers, seasonal affective disorder, and sleep disorders in the elderly.

This study is a double-blind, randomized, placebo-controlled, crossover, single-center study in healthy young men. After completing the screening visit, subjects follow a regular sleep/wake schedule for 2 weeks at home while wearing an actigraphy monitor in order to confirm their compliance. After the 2-week period, subjects begin the in-laboratory portion of the study, during which they will spend a baseline day in constant routine (CR) conditions, a night of sleep in the laboratory, followed by another CR day and night, during which time subjects will not go to sleep, but have a melatonin suppression test, followed by a day of recovery when subjects sleep and then are discharged before nighttime. Two hours before their typical bedtime on Day 2, subjects receive orally either the neuropeptide Y Y5 antagonist L-152,804 or placebo. Four hours later, 2 hours after their typical bedtime, subjects are exposed to a 5-hour pulse of moderately bright light (300-900 lux). Following the light exposure, subjects stay in CR conditions for another 5 hours, be allowed to sleep, and then leave the laboratory. Blood sampling is performed on subjects throughout the in-laboratory visit in order to collect samples for melatonin assays. Subjects remain at home for a 3- to 8-week washout period before returning for the second part of the study. During the last 2 weeks before the subjects come back into the laboratory, they follow a regular sleep/wake schedule for 2 weeks at home with actigraphic monitoring in order to confirm their compliance. Subjects then return to the laboratory to follow the same 3-day protocol but they receive the opposite drug treatment (i.e. neuropeptide Y Y5 antagonist or placebo). The primary response, suppression of melatonin, is assessed by calculating the percent change from baseline in the melatonin plasma during the 5-hour light pulse. The baseline value is defined as the melatonin plasma AUC of the corresponding 5 hours which occurred 24 hours earlier.

The administration of an effective neuropeptide Y Y5 antagonist can induce a change in the phase of the free-running circadian clock and block the phase-shifting effects of light on the mammalian circadian clock.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, effective dosages, other than the particular dosages as set forth herein above may be applicable as a consequence of variations in the responsiveness of the mammal being treated for any of the indications with the compounds of the invention indicated above. Likewise, the specific pharmacological responses observed may vary according to and depending upon the particular active compounds selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

1. A method for achieving a circadian rhythm phase-shifting effect in a mammal which comprises administering to the mammal an effective amount of a neuropeptide Y Y5 antagonist of structural formula: 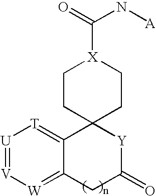
wherein A is selected from the group consisting of aryl or heteroaryl, wherein said aryl and heteroaryl groups may be optionally substituted on either the carbon or hetero atom, the substituent being selected from the group consisting of halogen, nitro, lower alkyl, halo(lower)alkyl, hydroxy(lower)alkyl, cyclo(lower)alkyl, lower alkenyl, lower alkoxy, halo(lower)alkoxy, lower alkylthio, carboxyl, lower alkanoyl, lower alkoxycarbonyl, lower alkylene optionally substituted with oxo, and a group represented by formula of-Q-D;
D is selected from the group consisting of aryl or heteroaryl, wherein said aryl and heteroaryl groups may be optionally substituted, the substituent being selected from the group consisting of halogen, cyano, lower alkyl, halo(lower)alkyl, hydroxy(lower)alkyl, hydroxy, lower alkoxy, halo(lower)alkoxy, lower alkylamino, di-lower alkylamino, lower alkanoyl and aryl; n is an integer from 0 to 1;
Q is selected from the group consisting of a single bond or carbonyl;
T, U, V and W are each independently selected from the group consisting of nitrogen or a methylene group, said nitrogen or methylene group may be optionally substituted with a substituent selected from the group consisting of: halogen, lower alkyl, hydroxy, and lower alkoxy;
X is selected from the group consisting of methylene or nitrogen;
Y is selected from the group consisting of nitrogen and oxygen, wherein said nitrogen may be optionally substituted with lower alkyl or oxygen;
and the pharmaceutically acceptable salts and esters thereof:

2. A method for resetting the internal circadian clock in a mammal which comprises administering to the mammal an appropriate amount of a neuropeptide Y Y5 antagonist, or a pharmaceutically acceptable salt or ester thereof.

3. A method for shortening the time of reentrainment of circadian rhythms in a mammal following a shift in the sleep-wake cycle which comprises administering to the mammal an appropriate amount of a neuropeptide Y Y5 antagonist, or a pharmaceutically acceptable salt or ester thereof.

4. A method for alleviating a circadian rhythm disorder in a mammal which comprises administering to the mammal an effective amount of a neuropeptide Y Y5 antagonist of structural formula: 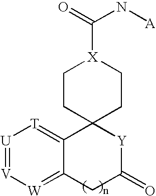
wherein A is selected from the group consisting of aryl or heteroaryl, wherein said aryl and heteroaryl groups may be optionally substituted on either the carbon or hetero atom, the substituent being selected from the group consisting of halogen, nitro, lower alkyl, halo(lower)alkyl, hydroxy(lower)alkyl, cyclo(lower)alkyl, lower alkenyl, lower alkoxy, halo(lower)alkoxy, lower alkylthio, carboxyl, lower alkanoyl, lower alkoxycarbonyl, lower alkylene optionally substituted with oxo, and a group represented by formula of -Q-D;
D is selected from the group consisting of aryl or heteroaryl, wherein said aryl and heteroaryl groups may be optionally substituted, the substituent being selected from the group consisting of halogen, cyano, lower alkyl, halo(lower)alkyl, hydroxy(lower)alkyl, hydroxy, lower alkoxy, halo(lower)alkoxy, lower alkylamino, di-lower alkylamino, lower alkanoyl and aryl;
n is an integer from 0 to 1;
Q is selected from the group consisting of a single bond or carbonyl;
T, U, V and W are each independently selected from the group consisting of nitrogen or a methylene group, said nitrogen or methylene group may be- optionally substituted with a substituent selected from the group consisting of: halogen, lower alkyl, hydroxy, and lower alkoxy;
X is selected from the group consisting of methylene or nitrogen;
Y is selected from the group consisting of nitrogen and oxygen, wherein said nitrogen may be optionally substituted with lower alkyl or oxygen;
and the pharmaceutically acceptable salts and esters thereof.

5. A method for the treatment of a circadian rhythm disorder in a mammal which disorder is selected from the group consisting of: time-zone change (jet-lag) syndrome, shift-work sleep disorder, delayed sleep-phase syndrome, advanced sleep-phase syndrome, and non-24-hour sleep-wake disorder which comprises administering to the mammal an effective amount of a neuropeptide Y Y5 antagonist of structural formula: 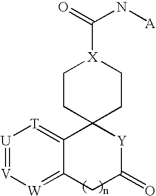
wherein A is selected from the group consisting of aryl or heteroaryl, wherein said aryl and heteroaryl groups may be optionally substituted on either the carbon or hetero atom, the substituent being selected from the group consisting of halogen, nitro, lower alkyl, halo(lower)alkyl, hydroxy(lower)alkyl, cyclo(lower)alkyl, lower alkenyl, lower alkoxy, halo(lower)alkoxy, lower alkylthio, carboxyl, lower alkanoyl, lower alkoxycarbonyl, lower alkylene optionally substituted with oxo, and a group represented by formula of -Q-D;
D is selected from the group consisting of aryl or heteroaryl, wherein said aryl and heteroaryl groups may be optionally substituted, the substituent being selected from the group consisting of halogen, cyano, lower alkyl, halo(lower)alkyl, hydroxy(lower)alkyl, hydroxy, lower alkoxy, halo(lower)alkoxy, lower alkylamino, di-lower alkylamino, lower alkanoyl and aryl;
n is an integer from 0 to 1;
Q is selected from the group consisting of a single bond or carbonyl;
T, U, V and W are each independently selected from the group consisting of nitrogen or a methylene group, said nitrogen or methylene group may be optionally substituted with a substituent selected from the group consisting of: halogen, lower alkyl, hydroxy, and lower alkoxy;
X is selected from the group consisting of methylene or nitrogen;
Y is selected from the group consisting of nitrogen and oxygen, wherein said nitrogen may be optionally substituted with lower alkyl or oxygen;
and the pharmaceutically acceptable salts and esters thereof.

6. A method for alleviating the effects of jet lag in a mammal which comprises administering to the mammal an alertness increasing amount of a neuropeptide Y Y5 antagonist of structural formula: 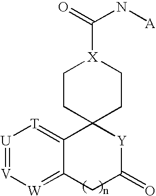
wherein A is selected from the group consisting of aryl or heteroaryl, wherein said aryl and heteroaryl groups may be optionally substituted on either the carbon or hetero atom, the substituent being selected from the group consisting of halogen, nitro, lower alkyl, halo(lower)alkyl, hydroxy(lower)alkyl, cyclo(lower)alkyl, lower alkenyl, lower alkoxy, halo(lower)alkoxy, lower alkylthio, carboxyl, lower alkanoyl, lower alkoxycarbonyl, lower alkylene optionally substituted with oxo, and a group represented by formula of -Q-D;
D is selected from the group consisting of aryl or heteroaryl, wherein said aryl and heteroaryl groups may be optionally substituted, the substituent being selected from the group consisting of halogen, cyano, lower alkyl, halo(lower)alkyl, hydroxy(lower)alkyl, hydroxy, lower alkoxy, halo(lower)alkoxy, lower alkylamino, di-lower alkylamino, lower alkanoyl and aryl;
n is an integer from 0 to 1;
Q is selected from the group consisting of a single bond or carbonyl;
T, U, V and W are each independently selected from the group consisting of nitrogen or a methylene group, said nitrogen or methylene group may be optionally substituted with a substituent selected from the group consisting of: halogen, lower alkyl, hydroxy, and lower alkoxy;
X is selected from the group consisting of methylene or nitrogen;
Y is selected from the group consisting of nitrogen and oxygen, wherein said nitrogen may be optionally substituted with lower alkyl or oxygen;
and the pharmaceutically acceptable salts and esters thereof.

7. A method for enhancing the quality of sleep in a mammal which comprises administering to the mammal an effective amount of a neuropeptide Y Y5 antagonist, or a pharmaceutically acceptable salt or ester thereof.

8. The method of claim 1 wherein the mammal is a human.

9. (canceled)

10. (canceled)

11. (canceled)

12. The method of claim 5 wherein the mammal is a human.

13. (canceled)

14. The method of claim 1 wherein the neuropeptide Y Y5 antagonist is administered in conjunction with melatonin or a compound which suppresses or stimulates melatonin production

15. The method of claim 1 wherein the neuropeptide Y Y5 antagonist is administered in conjunction with a compound which enhances sleep quality.

16. The method of claim 1 wherein the neuropeptide Y Y5 antagonist is administered in conjunction with light therapy.

17. The method of claim 5 wherein the neuropeptide Y Y5 antagonist is administered in conjunction with melatonin or a compound which suppresses or stimulates melatonin production.

18. The method of claim 5 wherein the neuropeptide Y Y5 antagonist is administered in conjunction a compound which enhances sleep quality.

19. The method of claim 5 wherein the neuropeptide Y Y5 antagonist is administered in conjunction with light therapy.

20. A method for the prevention of a circadian rhythm disorder in a mammal which disorder is selected from the group consisting of: time-zone change (jet-lag) syndrome, shift-work sleep disorder, delayed sleep-phase syndrome, advanced sleep-phase syndrome, and non-24-hour sleep-wake disorder which comprises administering to the mammal an effective amount of a neuropeptide Y Y5 antagonist of structural formula: 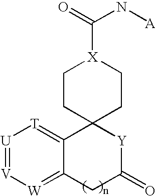
wherein A is selected from the group consisting of aryl or heteroaryl, wherein said aryl and heteroaryl groups may be optionally substituted on either the carbon or hetero atom, the substituent being selected from the group consisting of halogen, nitro, lower alkyl, halo(lower)alkyl, hydroxy(lower)alkyl, cyclo(lower)alkyl, lower alkenyl, lower alkoxy, halo(lower)alkoxy, lower alkylthio, carboxyl, lower alkanoyl, lower alkoxycarbonyl, lower alkylene optionally substituted with oxo, and a group represented by formula of -Q-D;
D is selected from the group consisting of aryl or heteroaryl, wherein said aryl and heteroaryl groups may be optionally substituted, the substituent being selected from the group consisting of halogen, cyano, lower alkyl, halo(lower)alkyl, hydroxy(lower)alkyl, hydroxy, lower alkoxy, halo(lower)alkoxy, lower alkylamino, di-lower alkylamino, lower alkanoyl and aryl;
n is an integer from 0 to 1;
Q is selected from the group consisting of a single bond or carbonyl;
T, U, V and W are each independently selected from the group consisting of nitrogen or a methylene group, said nitrogen or methylene group may be optionally substituted with a substituent selected from the group consisting of: halogen, lower alkyl, hydroxy, and lower alkoxy;
X is selected from the group consisting of methylene or nitrogen;
Y is selected from the group consisting of nitrogen and oxygen, wherein said nitrogen may be optionally substituted with lower alkyl or oxygen;
and the pharmaceutically acceptable salts and esters thereof.

21. A method for alleviating the effects of shift-work sleep disorder in a human in need thereof which comprises administering to the human an effective amount of a CNS-penetrating neuropeptide Y Y5 antagonist of structural formula: 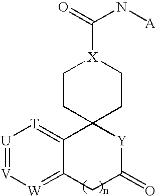
wherein A is selected from the group consisting of aryl or heteroaryl, wherein said aryl and heteroaryl groups may be optionally substituted on either the carbon or hetero atom, the substituent being selected from the group consisting of halogen, nitro, lower alkyl, halo(lower)alkyl, hydroxy(lower)alkyl, cyclo(lower)alkyl, lower alkenyl, lower alkoxy, halo(lower)alkoxy, lower alkylthio, carboxyl, lower alkanoyl, lower alkoxycarbonyl, lower alkylene optionally substituted with oxo, and a group represented by formula of -Q-D;
D is selected from the group consisting of aryl or heteroaryl, wherein said aryl and heteroaryl groups may be optionally substituted, the substituent being selected from the group consisting of halogen, cyano, lower alkyl, halo(lower)alkyl, hydroxy(lower)alkyl, hydroxy, lower alkoxy, halo(lower)alkoxy, lower alkylamino, di-lower alkylamino, lower alkanoyl and aryl;
n is an integer from 0 to 1;
Q is selected from the group consisting of a single bond or carbonyl;
T, U, V and W are each independently selected from the group consisting of nitrogen or a methylene group, said nitrogen or methylene group may be optionally substituted with a substituent selected from the group consisting of: halogen, lower alkyl, hydroxy, and lower alkoxy;
X is selected from the group consisting of methylene or nitrogen;
Y is selected from the group consisting of nitrogen and oxygen, wherein said nitrogen may be optionally substituted with lower alkyl or oxygen;
and the pharmaceutically acceptable salts and esters thereof.

22. The method of claim 21 wherein the neuropeptide Y Y5 antagonist is administered in conjunction with melatonin or a compound which suppresses or stimulates melatonin production.

23. The method of claim 21 wherein the neuropeptide Y Y5 antagonist is administered in conjunction with light therapy.

24. A method for the treatment of a sleep disorder in a human in need thereof which comprises administering to the human an effective amount of a CNS-penetrating neuropeptide Y Y5 antagonist of structural formula: 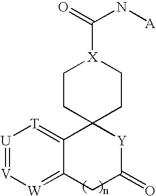
wherein A is selected from the group consisting of aryl or heteroaryl, wherein said aryl and heteroaryl groups may be optionally substituted on either the carbon or hetero atom, the substituent being selected from the group consisting of halogen, nitro, lower alkyl, halo(lower)alkyl, hydroxy(lower)alkyl, cyclo(lower)alkyl, lower alkenyl, lower alkoxy, halo(lower)alkoxy, lower alkylthio, carboxyl, lower alkanoyl, lower alkoxycarbonyl, lower alkylene optionally substituted with oxo, and a group represented by formula of -Q-D;
D is selected from the group consisting of aryl or heteroaryl, wherein said aryl and heteroaryl groups may be optionally substituted, the substituent being selected from the group consisting of halogen, cyano, lower alkyl, halo(lower)alkyl, hydroxy(lower)alkyl, hydroxy, lower alkoxy, halo(lower)alkoxy, lower alkylamino, di-lower alkylamino, lower alkanoyl and aryl;
n is an integer from 0 to 1;
Q is selected from the group consisting of a single bond or carbonyl;
T, U, V and W are each independently selected from the group consisting of nitrogen or a methylene group, said nitrogen or methylene group may be optionally substituted with a substituent selected from the group consisting of: halogen, lower alkyl, hydroxy, and lower alkoxy;
X is selected from the group consisting of methylene or nitrogen;
Y is selected from the group consisting of nitrogen and oxygen, wherein said nitrogen may be optionally substituted with lower alkyl or oxygen;
and the pharmaceutically acceptable salts and esters thereof.

25. A method for the prevention of a sleep disorder in a human in need thereof which comprises administering to the human an effective amount of a CNS-penetrating neuropeptide Y Y5 antagonist of structural formula: 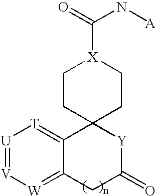
wherein A is selected from the group consisting of aryl or heteroaryl, wherein said aryl and heteroaryl groups may be optionally substituted on either the carbon or hetero atom, the substituent being selected from the group consisting of halogen, nitro, lower alkyl, halo(lower)alkyl, hydroxy(lower)alkyl, cyclo(lower)alkyl, lower alkenyl, lower alkoxy, halo(lower)alkoxy, lower alkylthio, carboxyl, lower alkanoyl, lower alkoxycarbonyl, lower alkylene optionally substituted with oxo, and a group represented by formula of -Q-D;
D is selected from the group consisting of aryl or heteroaryl, wherein said aryl and heteroaryl groups may be optionally substituted, the substituent being selected from the group consisting of halogen, cyano, lower alkyl, halo(lower)alkyl, hydroxy(lower)alkyl, hydroxy, lower alkoxy, halo(lower)alkoxy, lower alkylamino, di-lower alkylamino, lower alkanoyl and aryl;
n is an integer from 0 to 1;
Q is selected from the group consisting of a single bond or carbonyl;
T, U, V and W are each independently selected from the group consisting of nitrogen or a methylene group, said nitrogen or methylene group may be optionally substituted with a substituent selected from the group consisting of: halogen, lower alkyl, hydroxy, and lower alkoxy;
X is selected from the group consisting of methylene or nitrogen;
Y is selected from the group consisting of nitrogen and oxygen, wherein said nitrogen may be optionally substituted with lower alkyl or oxygen;
and the pharmaceutically acceptable salts and esters thereof.

26. The method of claim 24 wherein the neuropeptide Y Y5 antagonist is administered in conjunction with melatonin or a compound which suppresses or stimulates melatonin production.

27. The method of claim 24 wherein the neuropeptide Y Y5 antagonist is administered in conjunction with light therapy.

28. A method for the treatment of a sleep disorder in an elderly human in need thereof which comprises administering to the human an effective amount of a CNS-penetrating neuropeptide Y Y5 antagonist of structural formula: 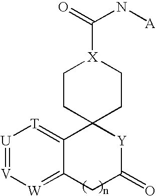
wherein A is selected from the group consisting of aryl or heteroaryl, wherein said aryl and heteroaryl groups may be optionally substituted on either the carbon or hetero atom, the substituent being selected from the group consisting of halogen, nitro, lower alkyl, halo(lower)alkyl, hydroxy(lower)alkyl, cyclo(lower)alkyl, lower alkenyl, lower alkoxy, halo(lower)alkoxy, lower alkylthio, carboxyl, lower alkanoyl, lower alkoxycarbonyl, lower alkylene optionally substituted with oxo, and a group represented by formula of-Q-D;
D is selected from the group consisting of aryl or heteroaryl, wherein said aryl and heteroaryl groups may be optionally substituted, the substituent being selected from the group consisting of halogen, cyano, lower alkyl, halo(lower)alkyl, hydroxy(lower)alkyl, hydroxy, lower alkoxy, halo(lower)alkoxy, lower alkylamino, di-lower alkylamino, lower alkanoyl and aryl;
n is an integer from 0 to 1;
Q is selected from the group consisting of a single bond or carbonyl;
T, U, V and W are each independently selected from the group consisting of nitrogen or a methylene group, said nitrogen or methylene group may be optionally substituted with a substituent selected from the group consisting of: halogen, lower alkyl, hydroxy, and lower alkoxy;
X is selected from the group consisting of methylene or nitrogen;
Y is selected from the group consisting of nitrogen and oxygen, wherein said nitrogen may be optionally substituted with lower alkyl or oxygen;
and the pharmaceutically acceptable salts and esters thereof.

29. A method for the prevention of a sleep disorder in an elderly human in need thereof which comprises administering to the human an effective amount of a CNS-penetrating neuropeptide Y Y5 antagonist of structural formula: 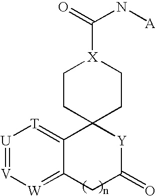
wherein A is selected from the group consisting of aryl or heteroaryl, wherein said aryl and heteroaryl groups may be optionally substituted on either the carbon or hetero atom, the substituent being selected from the group consisting of halogen, nitro, lower alkyl, halo(lower)alkyl, hydroxy(lower)alkyl, cyclo(lower)alkyl, lower alkenyl, lower alkoxy, halo(lower)alkoxy, lower alkylthio, carboxyl, lower alkanoyl, lower alkoxycarbonyl, lower alkylene optionally substituted with oxo, and a group represented by formula of -Q-D;
D is selected from the group consisting of aryl or heteroaryl, wherein said aryl and heteroaryl groups may be optionally substituted, the substituent being selected from the group consisting of halogen, cyano, lower alkyl, halo(lower)alkyl, hydroxy(lower)alkyl, hydroxy, lower alkoxy, halo(lower)alkoxy, lower alkylamino, di-lower alkylamino, lower alkanoyl and aryl;
n is an integer from 0 to 1;
Q is selected from the group consisting of a single bond or carbonyl;
T, U, V and W are each independently selected from the group consisting of nitrogen or a methylene group, said nitrogen or methylene group may be optionally substituted with a substituent selected from the group consisting of: halogen, lower alkyl, hydroxy, and lower alkoxy;
X is selected from the group consisting of methylene or nitrogen;
Y is selected from the group consisting of nitrogen and oxygen, wherein said nitrogen may be optionally substituted with lower alkyl or oxygen;
and the pharmaceutically acceptable salts and esters thereof.

30. The method of claim 28 wherein the neuropeptide Y Y5 antagonist is administered in conjunction with melatonin or a compound which suppresses or stimulates melatonin production.

31. The method of claim 28 wherein the neuropeptide Y Y5 antagonist is administered in conjunction with light therapy.

32-36. (canceled)
